(19) United States
(12) Patent Application Publication
(10) Pub. No.: US 2020/0276353 A1
(43) Pub. Date: Sep. 3, 2020

(54) DEVICE FOR DIFFUSING VOLATILE SUBSTANCES AND CARTRIDGE COMPRISING CAPSULES OF PERFUMING VOLATILE SUBSTANCES AND USABLE WITH SUCH A DEVICE OF ANY OTHER DEVICE COMPRISING A MECHANICAL OR VIBRATORY CAPSULE ACTUATOR

(71) Applicant: Kairos Capital S.A., Luxembourg (LU)

(72) Inventor: Alexandre Juving-Brunet, Erpeldange Sur Sure (LU)

(21) Appl. No.: 16/647,456

(22) PCT Filed: Sep. 14, 2018
(86) PCT No.: PCT/EP2018/074974

§ 371 Date: Mar. 13, 2020

(30) Foreign Application Priority Data
Sep. 16, 2017 (FR) 1770955
Sep. 16, 2017 (FR) 1770965
Sep. 21, 2017 (FR) 1770996
Sep. 21, 2017 (FR) 1770997

(51) Int. Cl. A61L 9/12; A61L 9/14

(52) U.S. Cl. CPC A61L 9/125; A61L 9/122; A61L 2209/132; A61L 2209/111; A61L 2209/133; A61L 9/14

(57) ABSTRACT

A device for the synchronized, smart and connected diffusion of perfuming volatile substances in the air, such as multiple fragrances and perfumes, and to manage the device according to outside conditions in time and space or according to virtual information provided by an external system, the device comprising a holder for receiving a cartridge comprising at least one capsule containing a liquid substance to be diffused, such as a perfumed or fragrant liquid, a motor designed to rotate the holder, a control module comprising motor control means to rotate the holder and diffusion control means comprising an actuator designed to act upon the capsules, causing them to diffuse the totality or part of the substance contained therein. The present disclosure also relates to a volatile substance cartridge designed to be detachably mounted on such a diffusion device or the like.

### CROSS-REFERENCE TO RELATED APPLICATIONS

This application is a national phase entry under 35 U.S.C. § 371 of International Patent Application PCT/EP2018/074974, filed Sep. 14, 2018, designating the United States of America and published as International Patent Publication WO 2019/057643 A9 on Mar. 28, 2019, which claims the benefit under Article 8 of the Patent Cooperation Treaty to French Patent Application Serial No. 1770955, filed Sep. 16, 2017, to Serial No. 1770965, filed Sep. 16, 2017, to Serial No. 1770996, filed Sep. 21, 2017, and to Serial No. 1770997, filed Sep. 21, 2017.

### TECHNICAL FIELD

The present disclosure relates to the field of diffusion of fragrant volatile sub stances.

The present disclosure more particularly relates to a device for diffusing volatile substances, and a cartridge of volatile substances usable with such a device or any device generating an air flow.

### BACKGROUND

The sense of smell is one of the preponderant human senses of survival (detection of dangers and threats), of life (food, pheromones, etc.) but also of social valence (the hedonistic character of perfumery, beauty, seduction, lifestyles, etc.). For a long time considered as a minor sense because of its analogies with the animal world (Socrates and more recently Freud or de Broca), its study has never been the subject of fundamental research leading to advanced industrial applications. The sense of smell through the organ known as the cribriform plate is responsible, in particular, for communicating the organoleptic chemical information of olfaction (the smell) and retro-olfaction (the taste).

On the other hand, attempts at olfactory synchronization in the 20th century were hampered (failures of ODORAMAX, ODORAVISION, etc.) by the lack of sufficiently precise techniques and technologies to manage the control of synthetic perfumes or fragrances in a flexible and directed manner due to their molecular nature, which excludes any precise control or even digitization process specific to visual and auditory information. Indeed, the image of a photonic nature is driven by light at 300,000 km/s, the sound of a vibratory nature by air at 340 m/s but the perfume is of a molecular (volatile) nature and must be driven by a dynamic of carrier fluids which have different propagation regimes depending on the flows and ambient atmospheric conditions.

It has been left for the time being in view of the difficulty of its implementation and, in particular, the fact that a fragrance must at the same time be adapted to the circumstance, must not persist outside these external circumstances and may be composite because it comes from several sources; moreover, under certain circumstances it is necessary to take into account external fragrances in order to modulate the level of the aromas which are associated in the composition of the desired fragrance. All these requirements require both a mastery of synthetic chemistry but also of many different technical fields such as fluid and molecular dynamics, which must be integrated into a portable and ergonomic device. The state of the art of all these technologies was previously not mature for execution according to the present disclosure.

At the beginning of the 21st century, the evolution of connected entertainment and multimedia, in particular, has led patent writers in this field to expand their writing by adding scent to the other senses, mainly sound and image, which are of course the ones used in the systems that have emerged over the last two decades. Position and location have also been the subject of numerous developments.

More recently, the patents for technologies marketed under the names SMELLIT© (trademark) and ODORAVISION© (trademark) offer audio-visual and olfactory coordination without offering an individual and portable solution integrated into virtual and augmented reality headsets, nor do they solve the problem of “olfactory mist” resulting from the accumulation in a room of any size of several strata of opposing fragrances with uncontrollable effects. More recently, the same phenomenon can be observed in articles on the future and in patent applications on the interactions between sensors or sensory transmitters that can be connected to remote servers, particularly using telephones. These patents as well as the application EP 2 632 191 only give fragmented indications on the practical realization of such remote interactions and, in particular, the key device for the production/emission/recovery of fragrant volatile molecules.

More recently, Ubisoft made a remarkable demonstration by disclosing a fragrance generation prototype coupled with its virtual reality devices. The device presented consists only of single fixed, manually removable containers, and offering only a limited range of fragrances with an essence that cannot be simultaneously associated with other audiovisual systems. The main interest of the approach in this context is the demonstration that feedback is interesting from the point of view of its interactivity between an action carried out by a person, or triggered by a film, and the image and/or sound “feedback” that can be received and or interpreted either directly by another player, or within the framework of a statistical analysis on behalf of film producers or cinema exhibitors in a non-invasive form, in particular, by simply measuring the accelerations linked to the scenes in the film and/or the fragrances diffused.

Application WO 03/088627, discloses a portable device capable of delivering a selected fragrance from a predetermined basket, but does not provide any specific or innovative information on the technique or the method used to allow the diffusion of fragrant molecules. It only refers to a fragrance generator (a “Scent generator”) without any further precision or technical details.

The application EP 2 777 718 discloses a perfume diffusing device, notably dedicated to motor vehicles. Such a device is inherently limited to the diffusion of a limited number of fragrances and is not designed for use in a wide variety of circumstances. The system has no innovations in terms of directed micro-diffusion and does not explain which fragrance generation principle it is based on. Moreover, the defined fragrances cannot be neutralized in the sense of fragrance destruction as proposed in the continuation of the present disclosure; but by masking; in particular, by saturation.

The patent filed in 2009 and listed under the reference WO 05/55459 describes a device for generating an original perfume from a cavity that is animated with a vibratory device. Such a device is not usable per se in the present disclosure, in particular, because industrially, it requires the realization of a means which is not standard. This device also involves chemical constraints due to the porosity of the system due to its non-occlusion, a problem that is outdated and solved by the present disclosure. Moreover, it is based on a principle of diffusion from mechanical oscillations which are not the subject of this patent. Other numerous disclosures that will not be mentioned exhaustively concern fragrance diffusion, most of the time focusing on an original technical achievement, such as applications or patents FR 2 944 705; FR 2 959 937; WO2013/076033; WO2014/182337; EP 2769862.

The application WO 2006/032866 describes a portable fragrance diffuser for use in the field of learning, it is mainly oriented on the design of this device at the mechanical and fluidic level, this description corresponds neither to the use which is envisaged, nor to the packaging envisaged in order to be able to be coupled in particular to a virtual reality headset.

The application WO 2014/154909 schematizes a fragrance diffusion system by the generation of an air or gas flow without precisely describing the mechanical, electrical or vibratory principle which is the subject of this patent and which is responsible for the generation of the diffusion. The size of the system presented also excludes any miniaturization.

The application EP 2 965 692 discloses one embodiment of a system comprising a fragrance generation device without description of the diffusion principle, in order to strengthen cognition processes and process memorization. The present disclosure does not describe the methods implemented and does not disclose the techniques used to diffuse, orient and control olfactory synchronization.

The application WO 2014/028372 discloses the use of a device for generating a smell from a multiplicity of fragrances, this device works from a gas flow that passes through a multiplicity of channels each corresponding to a smell and manages their relative intensity by closing a valve. This method has many drawbacks including fragrance persistence and can be used especially when it is necessary to reconstitute a smell from particular fragrances. If, for example, one wishes to generate more than a dozen fragrances for an advertising scenario, this device would have to carry hundreds of basic aromas to be able to generate them in the moment. This is contrary to the regulations on French and international IFRA (International Fragrance Association) certifications, which prohibit the diffusion of a scent without prior IFRA certification. It is therefore unsuitable for the industrial and commercial target which is that of the present disclosure, in particular because different moments will require a total break from one moment to the next, the process of realization of which is not described in the disclosure.

The application WO2015/043803 discloses a fragrance generator from a gas flow that comes to lick successively several fragrance-emitting compartments and which can be closed or opened but with simultaneous diffusion. This solution is not adapted to the calibrated management of fragrance diffusion as are the patent-pending head-sprays which allow to calculate precisely the quantity of fragrant liquid emitted. Moreover, the device excludes a multiplicity of fragrances taken separately: no directed or selected flow is detected that is capable of emitting several fragrances according to a pre-established or controlled sequence.

Application WO2015/145453 describes a device driven by a computer or a microprocessor, and a method suitable for the dissemination of fragrances related to the physical presence or occurrence of an expected event in a specific physical area.

It schematizes only the global synchronization system without any technical diffusion application.

The application WO2016/034131 describes a device which makes it possible to increase, with fragrances, the information transmitted by adaptable earphones either on a telephone or on a computer, in the same way the application WO2016/147544 or the application patent no EP1570868 introduces the same application for a diffusion by an air-conditioning or ventilation system. These applications do not describe any state of the art of the mode or principle of diffusion. They only deal with the environment in which the technique is used.

Similarly, the application EN 3007540 describes a device that allows a fragrance alarm clock with an exchangeable cartridge, this device addresses only a limited event and a dose of essential oil subject to a flow of air. This device is therefore proprietary and is not suitable for circumstantial use related to interaction with external events.

The application WO2015/080117 discloses the realization of a fragrance generator from dry extract by a forced flow of air through chambers containing the extracts. Such a device is not adapted to the realization of certain smells and it is more difficult to implement the realization of custom-made smells, thus describing, for example, a particular situation related to an event described by words or images in such a packaging. The device does not allow a fine calibration of the olfactory emission specific to the directed micro-emission object of the deposit. Moreover, the described device does not show how it can eliminate fragrances, because the remanence also exists with the dry extracts and the particles, which are temporarily fixed in a corner can then be released and come to give the following moment a polluted fragrance because completely unsuitable with the new scene. This is one of the reasons that led us to develop a device that is both easy to feed with industrial containers usually used by perfumers in particular and that also allows the choice of either mixing several smells, or eliminating the smell used to use another, completely different one, and perfectly adapted to a new context, all these operations being electronically managed and in relation to information from connected objects or other also connected actors.

Patent application No. US2010/044460 A1 is based on a piezoelectric diffusion principle claimed to be able to diffuse a precisely calibrated fragrance. This technical principle is not retained or followed by the present disclosure because of the porosity of the system, which could not abstract the tank and its fragrant contents from the surrounding air. A mixture pollution effect is to be expected in the case of multi-perfume diffusion.

Application WO 2014/154909 is based on an olfactory cartridge principle but does not specify how the carrier gas line is managed or how the carrier gas reaches an individual's nasal cavity.

The purpose of the present disclosure described hereafter is to solve the problems and limitations of the prior art as cited in the state of the art comments: there is no autonomous or structural (fixed) portable device that allows the generation or synthesis of a composite ambiance of linked and adapted fragrances to external circumstances, in particular by means of connected objects through advertising media, connected functional objects or a relative position established by an electronic system or remote applications or applications integrated in the device itself. Moreover, existing devices are not quickly adaptable at the industrial level for the use of existing perfume packaging and use only one, often proprietary, means. Moreover, the problem of fragrance persistence and the management of user perception is not taken into account, whether it is for the management of the sensitivity of the device to ambient fragrances or for the analysis of a customer or consumer's reaction.

### BRIEF SUMMARY

To this end, and according to a first aspect, the present disclosure proposes a device for the synchronized, smart and connected diffusion of perfuming volatile substances in the air, such as multiple fragrances and perfumes and to manage the device according to outside conditions in time and space or according to virtual information provided by an external system, the device comprising:

In a preferred configuration, the actuator comprises a cylinder designed to axially actuate the capsule in the form of an assembly

a holder for receiving a cartridge comprising at least one capsule containing a fragrant liquid substance to be diffused such as a perfumed or fragrant liquid

a motor designed to rotate the holder,

a control module comprising motor control means to rotate the holder and diffusion control means comprising an actuator designed to act upon the capsules causing them to diffuse the totality or part of the substance contained therein.

In a preferred configuration, the actuator comprises a cylinder designed to axially actuate the capsule in the form of a tube/spray-pump assembly which, under the compression of the micro-cylinder, nebulizes the substance contained therein.

It can also be provided that the cylinder is an electrically controlled micro-cylinder, possibly controlled by a solenoid valve with an air supply regulated by a single- or double-rod pressure regulator, which is responsible for pressing each capsule into its housing according to a programmed sequence. Advantageously, the actuator is a vibratory actuator designed to produce ultrasound to achieve atomization of the liquid substance contained in cartridges so as to allow ultrasonic nebulization of the substance.

Advantageously, the diffusion device has at least one micro-fan 9 designed to pulsate hot air or gas into the cartridge when the cartridge is mounted on the device via the holder.

Advantageously, the diffusion device comprises a heating resistor and/or means exerting an undulatory stress associating at least two asynchronous frequencies, at least one of which is at least non-linear. This makes it possible to act on the duration of the diffusion: to be included in the description.

Advantageously, the diffusion device comprises a pair of air extraction fans designed to create a zone of air depression in front of a user's face so as to create an individual perfuming zone and to avoid any secondary pollution and contiguous to a device located nearby.

Advantageously, the diffusion device comprises a module for collecting information from an external means connected or previously loaded in the memory and information from sensors or means catching the close environment around the user and, simultaneously or in a programmed way, combining them to determine the choice and duration of diffusion of the substance to be diffused.

Advantageously, the diffusion device has an interface in the form of a digital and touch screen for the control by a user of the functionalities of the device.

Advantageously, the diffusion device includes a camera for monitoring and collecting information.

Advantageously, the diffusion device has an electronic, magnetic or inductive connection associated with the micro-motor to interact in terms of synchronization of perfumes and user's reaction with an external control system.

Advantageously, the diffusion device has a first communication interface designed to allow bidirectional communication with a remote diffusion management server.

Advantageously, the diffusion device is portable.

The present disclosure also relates to a cartridge of volatile substances adapted to be removably mounted on a diffusion device as described above or any other device comprising a mechanical or vibratory capsule actuator, wherein it comprises a plurality of housings adapted to receive, respectively, a capsule containing a liquid substance to be diffused, each housing comprising an outlet opening for the substance diffused by the capsule placed in the housing in response to a request from the actuator of the diffusion device when the cartridge is mounted on the diffusion device.

According to a preferred embodiment, each housing is provided with a passage allowing a micro-cylinder constituting the actuator of the diffusion device to axially actuate the capsule in the form of a tube/spray-pump assembly received in the housing and which, under the effect of the compression of the micro-cylinder, nebulizes the substance it contains.

Advantageously, the cartridge comprises a plurality of channels having, respectively, an inlet opening and an outlet opening designed to allow the passage of an air flow generated by the device up to the level of the outlet opening of the housing that they are associated with, the inlet and outlet openings of each of the channels being independent of one another. This has the advantage of not polluting by the effect of remanence due to the autonomy of each channel.

Advantageously, the cartridge contains an RFID (Radio Frequency Identification) or NFC (Near Field Communication) chip.

Advantageously, the cartridge is designed to collect information from an external means connected or previously loaded in the memory and, simultaneously or in a programmed manner, information from sensors or means for catching the close environment around the user, and to establish, from the information, the diffusion context from which the choice of the capsule and the duration of diffusion of the substance contained in the capsule are determined.

Advantageously, the cartridge comprises at least one capsule containing a perfumed substance to be diffused and at least one capsule containing a product neutralizing the fragrance left by the perfumed substance or any other substance.

Advantageously, the cartridge has a cavity or channel within which the desired quantity of the selected perfume or fragrance is diffused, the cavity or channel being made of or surface-coated with an adsorbent material having water-repellency characteristics such that any proportion of the diffused fragrance quantity does not deposit on the duct.

The present disclosure further relates to a system for managing the diffusion of fragrance in an environment in response to a user control action comprising at least one diffusion device as previously described, capable of communicating with a first processing unit of a remote server, at least one cartridge as previously described and removably attached to the diffusion device, and a fragrance diffusion control interface connected to the processing unit.

In a preferred configuration, the management system comprises at least one image acquisition arrangement designed to photograph or film the user at the time of diffusion of the perfume in response to a command to diffuse the latter, the acquisition arrangement being connected to a second image processing unit to establish a behavioral profile related to the diffused perfume from at least the acquired images.

Advantageously, the control interface is integrated into the diffusion device.

### BRIEF DESCRIPTION OF THE DRAWINGS

Other objects and advantages of the present disclosure will appear in the following description, with reference to the attached drawings, in which:

FIG. 1 represents schematic views, respectively, of the front (a), back (b), in perspective (c) and top (d) of a device for diffusing volatile substances according to a first embodiment of the present disclosure;

FIGS. 2 and 4 to 6 represent exploded views of the diffusion device according to FIG. 1;

FIG. 3 represents a detailed view of the part A;

FIGS. 7 and 8 represent exploded bottom and top views of the perfume cartridge;

FIG. 9 represents schematic views, respectively, of the side (a), front (b), perspective (c) and top (d) of a device for diffusing volatile substances according to a second embodiment of the present disclosure;

FIGS. 10 to 12 represent exploded views of the diffusion device according to FIG. 8 from the front, back and side;

FIG. 13 represents an assembled view of the part A;

FIG. 14 represents a view of the implementation of the diffusion device of FIG. 9;

FIG. 15 represents schematic views, respectively, of the side (a), front (b), in perspective (c) and top (d) of a device for diffusing volatile substances according to a third embodiment of the present disclosure;

FIGS. 16 to 18 represent exploded views of the diffusion device according to FIG. 15 from the front and the back;

FIGS. 19 and 20 represent detailed views of the portion of part A from the front and the back;

FIG. 21 represents an exemplary architecture of a fragrance diffusion management system comprising a diffusion device according to the present disclosure and a volatile substance cartridge.

For greater clarity, the same or similar elements of the different embodiments are marked by identical references on all the figures.

### DETAILED DESCRIPTION

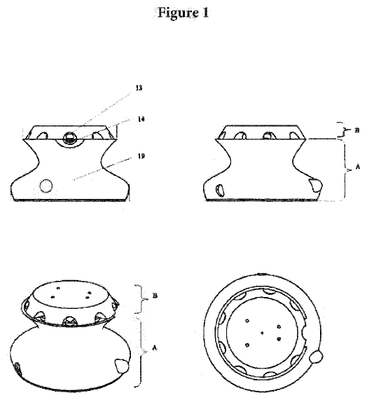

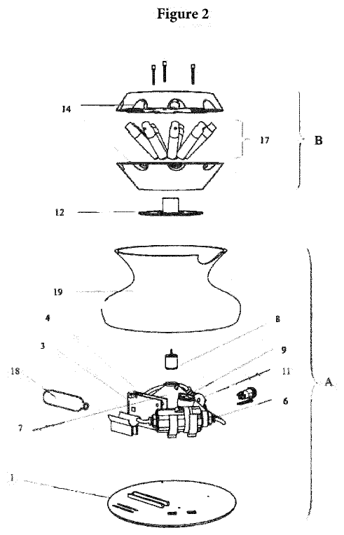

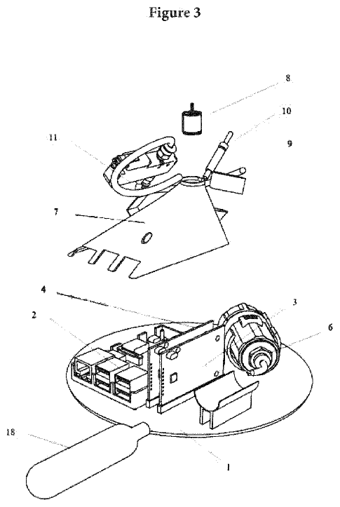

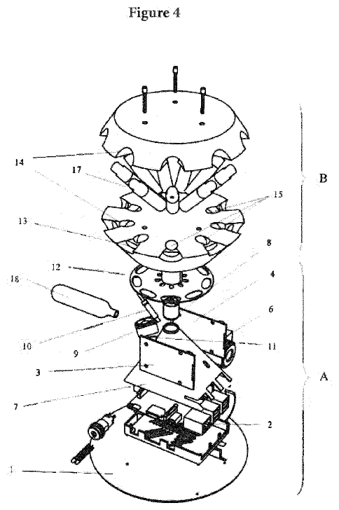

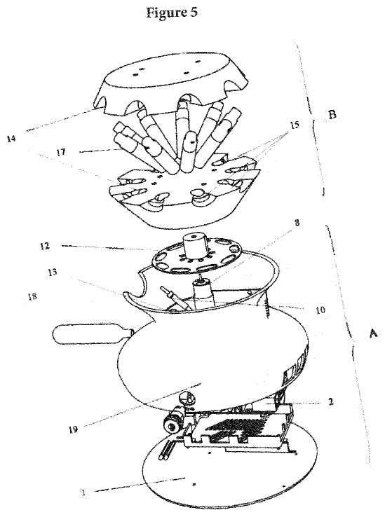

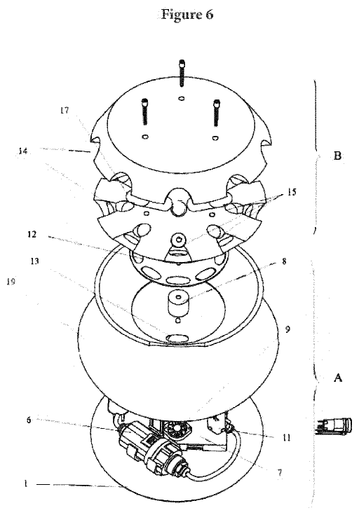

In relation to FIGS. 1 to 6, a device for diffusing volatile substances into the air is described, and more particularly a functional device for selecting, actuating and then diffusing a perfumed or fragrant liquid encapsulated and designed in a refillable or consumable multi-capsule cartridge.

These mechanical actions are synchronized by an autonomous on-board electronic interface or a remote connectable interface. It allows, in relation to a predefined programmed scenario, or by receiving any synchronization information from a software, application or connected intelligence, to increase the degree of immersion of customers, patients or collaborators by making the scenes more realistic, in particular through the use of messages or olfactory stimuli either as a support for the other senses usually used, or in a new way as an information device. The multiple applications concern the improvement or even the transformation of the sensory perception of individuals by acting on their psyche and in particular the organoleptic interface limbic system/Limbocampus/NeoCortex system responsible in particular for the human memorization process through the chemical encoding of information created by an electro-chemical discharge process created by the olfactory neurons (NO) receptors of the fragrant volatile molecules in the air carrying olfactory (smell) and retro-olfactory (taste) information. This action of multi-nebulization and/or synchronized and micro-diffused atomization is carried out toward the human nasal apparatus of the following user families:

Clients (Entertainment and advertising industries, food industry, tourism . . . ).

Collaborators (Immersive Distance Learning Sessions).

Patients (Medical treatment of all neurological disorders including phobias and Alzheimer's disease).

The device described hereafter (named “SNIFFY”) can be used to equip or complete audio, audio-visual, immersive audio-visual Virtual Reality or equivalent headsets, audio-visual with augmented reality display or equivalent, in vehicle interiors, cockpits or collective or individual equipment for airliners, public transport, urban structures such as intelligent street lamps, smart bollards or augmented and connected audio-visual and tactile panels, seats for opera, concert or cinema entertainment halls.

The device is a portable autonomous or structural (fixed) device that allows the generation or synthesis of a composite ambiance of linked fragrances and adapted to external circumstances, in particular by objects connected through advertising media, connected functional objects or a relative position established by an electronic system or remote applications or integrated into the device itself.

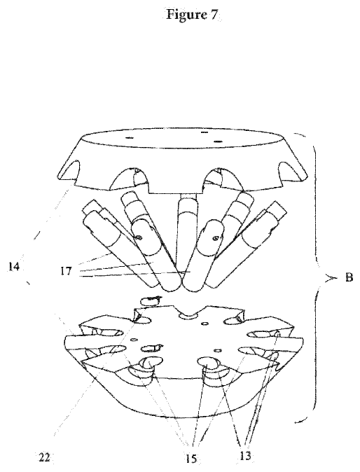

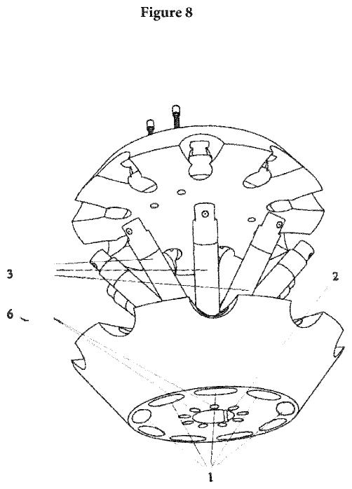

The device consists of two main parts A and B, with part A being the basis of the smart electromechanical system and part B being an inter-operably connected olfactory cartridge. The cartridge, shown in more detail in FIGS. 7 and 8, will be described in detail below.

In the embodiment described, the part A, which is the basis of the smart electromechanical system, comprises: a fixing plate 1 advantageously housing a motherboard 2 with a processor, a random access memory, several mechanical connections such as USB (Universal Serial Bus) and HDMI (High Definition Multimedia Interface) and Micro SD (Secure Digital) and a WIFI (Wireless Fidelity) card in accordance with the IEE 802.11 standard or higher, an RFID (Radio Frequency Identification) card 3, an NFC (Near Field Communication) card 4, a 5V power supply 5, an AUTOMAX type pressure regulator 6.

The mounting plate is surmounted by a frame 7 for mounting the following electromechanical components:
a micro-motor 8 for rotating a plate 12 (described below), which the cartridge is docked on. In the example described, the plate is a male plate. The micro-motor is advantageously selected for its energy consumption (e.g., 0.25 A/2V) and the as low as possible (e.g., less than 20 dB) noise induced by its operation, and in particular stepper motors corresponding to the two-phase family/20 per revolution/1.6 mNm/such as those marketed under the brand names Precistep©, FAULHABER© or equivalent supporting 0.3N or equivalent on its shaft, controlled by an optical encoder with a 2-channel digital output with at least 30 pulses per revolution,
an axial micro-fan 9 advantageously selected for its energy consumption limited to 5 Volt (V) and 0.1 Ampere (A) and a noise induced by its operation below 18 dB (A) and ensuring an air flow of a minimum of 18 pascal (Pa) corresponding to 2.20 m3/hr and allowing a double use of thermal extraction of the element 13 and of hot air or gas supply toward the collector 14;
a micro-cylinder 10 to actuate the capsules housed in the cartridge. The micro-cylinder is advantageously electrically controlled and air supply regulated by a pressure regulator 6, which can be single-acting or multiple-acting with a diameter between 02.5 mm and 025 mm, with standard or pneumatic damping, and equipped with magnetic limit switches of the Reed-Ils type or “Hall” effect according to the ISO 6432 standard, developing a speed of 0.3 to 0.7 ms and ensuring an adjustable pressure between 3.00 Bar and 7.00 Bar.
a solenoid valve 11 to control the micro-cylinder. The valve is advantageously an electronic pressure control valve with a valve diameter of 02.5 mm or more with an effective area of 1.3 mm3 or more with a response time of 6 ms.
means for controlling the micro-cylinder, such as a compressed air cartridge 18 or any other pressure supply to feed the micro-cylinder.

Cartridge or JOCC. The cartridge allows the sequential use of several perfumes or fragrances. It has compartments that can hold capsules, vials or cartridges of fragrant products in a liquid form. It is designed to be fixed on the part A described above, and more particularly on the male plate. This cartridge, which is shown in FIGS. 7 and 8, that can be consumable or refillable, includes, in the embodiment shown:
a circular base element housing a variable sequence of several housings 15 of variable number, perforated from below and designed circularly around a hollow central hub 16, which allows a female part of part A covered with a protective skirt or a motor base as described above to be snapped onto the micro-motor. The housings are designed to receive capsules 17, advantageously made of glass or polymer or metal material and composed of an independent spray-pump comprising, in their spray heads, a return spring allowing the return to the initial position after action of the micro-cylinder 10. Each capsule is inserted into a respective housing by its base, with the spray head extending opposite a dedicated collection space 14 (also referred to as a collector).
Hot air extraction corridors (e.g., ducts) 13 allowing the air flow to come, in the terminal part, into the collection space 14, which is the main area of exchange and of gas mixing between the air or the hot pulsed gas and the perfumed liquid nebulized at the pump-spray outlet.
In the case of cartridges of the “vibratory” type, these are associated with an interface enabling the product to be isolated or brought into contact with the diffusion component by an undulatory mechanism, usually but not restrictively ultrasound, the “host system” by its electronics instantaneously causes the emission of a fragrance in the form of a liquid.

It can be advantageously provided for the cartridge to be equipped with an RFID microchip allowing the traceability of the cartridge and its recognition by the base (Part A) of the diffusion device and powered by an internal power source such as a 1.5V or higher round micro-battery.

According to a preferred version of the present disclosure, the device according to the present disclosure is characterized in that cartridges of the “mechanical” and “Vibratory” type can be used simultaneously. This has the advantage of associating an important but fleeting instantaneous diffusion of a fragrance by the vibratory mode (for example, light like a perfume) with a persistent background fragrance.

According to an improvement to the present disclosure, it is essential, when a fragrance has been diffused by the device previously described, to be able to eliminate its residual action and the deposits of volatile particles deposited during the diffusion action. Ideally, the desired properties are to be able to progressively reduce its action, as already described by certain methods such as closing valves that isolate the source or shutters that divert the outlet away from the user's direction. A simple multi-channel solution is presented here, which prevents the effect of remanence, the diffusion of the product being obtained from the propulsion of a hot gas by a micro-fan 9 coming into contact with a nebulized perfumed liquid, which is then carried by the hot air/hot gas vector to a human olfactory apparatus or a targeted environment. The fixed quantity of perfume is delivered at the terminal part of the system thanks to the action of the micro-cylinder 10, which compresses by longitudinal thrust the bottle containing the fragrant liquid that passes from a liquid state to a semi-gaseous (or nebulized) state by the vacuum action of the Spray-Pump or “Venturi Effect”.

According to an advantageous version of the present disclosure, the duration for which the release of the fragrance can be modulated is obtained by adjusting the temperature by means of a heating resistor and/or an undulatory stress combining at least two asynchronous frequencies, at least one of which is linear, of a mechanical nature applied either at the level of the substrate in or on which the fragrant product is adsorbed or deposited, or at the level of the air stream, which transports the evaporated perfume to the user's nose, or in both these media. The choice of the mode of action, the chosen frequencies and the heating power can advantageously be selected by the microprocessor or part of a dedicated electronic component, with the help of a program linked to a knowledge base and/or the diffusion laws, notably those resulting from experimentation with the selected material and each of the perfumes or fragrances.

According to a preferred version of the present disclosure, the destruction of the fragrance may be carried out by the use of a dedicated capsule installed in the cartridge or in the specific system containing a perfume neutralizer, deodorant or oxidizer. Its composition and dosage will take into account not only the time constant of its reaction with fragrances, but also the fact that this product must not present any prophylactic danger or side effects on the user. This product cartridge can be used in addition to bactericidal, antifungal or virucidal products, which will also ensure good hygiene and maintenance of the device. In particular, products can be used for their action such as hydrogen peroxide at very low concentration, i.e., less than 2%, and terpene derivatives or essential pine oils at very low concentration. This description of usable products is not restrictive and may change according to the fragrances to be neutralized.

In a version preferred by the inventor, the device operates by the insertion of one or more part B cartridge(s) equipped with vials, which may be refillable and which may be removable and replaceable. The body of the cartridge is driven by the micro-motor, preferably step by step, allowing each capsule to be positioned along the axis corresponding to a continuity with the chamber/collector of the fragrant product. Depending on the circumstances determined by the interaction of a given fragrance with a synchronous event, an electronic device such as a processor or electronic component having a memory storage capacity and a dedicated program execution capacity, which can combine a user part and a part specific to hardware management or software related to hardware management, executes a program that determines the sequencing and the duration of emission of the liquid perfume capsules. The information on the contents of the cartridge and individually for each of the perfumes or fragrances that each barrel contains can be read by a method according to the state of the art and, in particular, by an optical code or RFID chip reading, which allows the above-mentioned program present in the electronics of the base of the system to control the sequencing or synchronization of the fragrance emissions. The activation of the micro-cylinder by the integrated electronics is carried out during the sequencing process or after detection of an event or external sign through one of the interfaces of the device according to the following procedures:
The device, in a nomadic or structural version, is capable of providing at least 500 pulses for the micro-cylinder while ensuring coordination with information/messages, i.e., 500 olfactory coordination actions called “SNIFF” with audio-visual stimuli or information with the capacity to actuate two cartridges simultaneously, each comprising, more or less depending on the structural or nomadic version, nine or more capsule housings containing approximately 1.5 ml or more of perfumed liquid for each capsule.
Each dose of nebulized or atomized perfume corresponds to 0.05 ml plus or minus 50% as the margin of error.

The micro-cylinder 10 performs a longitudinal or axial thrust on a capsule and causes the pressure rise of a capsule 17 of perfume or fragrant chemical product placed in its housing 15. At the end of its stroke, the micro-cylinder stops its action against the cartridge stop, the return spring in the spray pump limiting the volume dose produced for each action and ensuring the cartridge returns to its initial position. The action of the micro-cylinder is a longitudinal action according to a single- or double-acting rod. The action, electronically controlled by the solenoid valve 11 and supplied with compressed air from the compressed air cartridge 18 or any other pressure supply, consists in pushing a piston compressing the spray cap against the stop on the buttress of the cartridge. Each capsule is held in its housing by a return spring allowing a return to the initial position once the action of the cylinder has been carried out. The relaxation phase caused by the return spring causes a depression in the liquid perfume capsule and the perfume cartridge then releases a predefined unit dose. The perfume or fragrance is dispersed by the “Venturi Effect” in the form of small liquid droplets (nebulization) in a slit opening onto a chamber/collector or olfactory channel 14, which stores the perfume transformed into droplets carried by the gas vector created by the micro-fan, simultaneously actuated with the ejection of the perfume dose. This action can be performed several times on the same capsule according to the established programming or the sequence order sent by the CPU, the motherboard or any other on-board electronic device.

The air flow created carries the olfactory molecules contained in the chamber/collector, which releases the olfactory molecules as close as possible to the nasal apparatus of the customer, collaborator or patient. This targeted micro-nebulization has several new advantages:
Firstly, micro-nebulization management allows the calibration of a reduced volume of olfactory liquid with the effect of not saturating the human nasal system.
Secondly, the micro-nebulization vector placed directly under the nasal apparatus avoids the olfactory saturation effect of the atmosphere immediately surrounding the patient/customer/collaborator to help individualize the olfactory experience.
Thirdly, this non-saturation of the nasal system opens the possibility of multiple action over a temporal window of variable duration, diminishing or repelling the saturation effect of the human nasal system. It provides approximately 20 different synchronized olfactory stimuli over a 120-minute time span, while the human nasal system reaches saturation after 30 minutes of sustained use.

The part B cartridge can hold a variable number of capsules. These capsules are equipped with identification means such as an optical, electronic or RFID identifier, which both ensures that the cartridge matches the intended situation or sequence and then matches a specific vial with an intended situation in the sequence.

The motherboard 2 authenticates, controls engine rotations and stops, drives the actions of the cylinders, the synchronization of the fan and the management of the electronic or programmatic information entering and leaving the system. The CPU is also the transmitter and receiver of messages and codes to the gateways outside the system (“Clouds” and servers) responsible for managing the fleet of all the synchronized systems deployed.

The motherboard 2 transmits and receives information to the servers outside the present system in charge of driving the fleets of deployed systems.

All the electrical components of the device according to the present disclosure are powered by a battery, in particular, the actions of selecting by rotation of the cartridge and producing a dose of liquid through the cylinder, the electronics, the control interface and its possible signaling devices (operation diodes, for example).

According to an advantageous embodiment of the contextual synchronous fragrance diffusion device according to the present disclosure, the diffusion context, which determines the choice and the duration of diffusion of a perfume or fragrance, can advantageously combine information from an external means connected or previously loaded in the memory and simultaneously or in a programmed manner information from sensors or means for catching the close environment around the user. In particular, the device may be equipped with sensors allowing, inter alia, the transfer of RFID data or data contained in other contactless devices distributed in open or closed environments communicating with the nomadic system or its structural version by known contactless means (radio such as Bluetooth, WiFi or other systems operating by induction).

Advantageously, the diffusion device includes a camera, which makes it possible, in particular, to read an optical support either by text recognition, shape recognition or by reading an optical code such as a bar code. Such a device according to the present disclosure can also take advantage of the integration in the electronics of any system that allows the identification of a situation such as for example a positioning or an identification by reading an RFID medium, for example, these modes of identification of the contextual situation not being restrictive.

A preferred version of the Contextual Synchronous Odor Diffusion Device (CSDD) is a device that has an electronic connection so that it can interact in terms of perfume synchronization and user response with remote computers and programs on the global network.

According to a preferred version of the contextual synchronous fragrance diffusion device, the cylinder used is a single- or double-rod electric micro-cylinder in charge of pressing each capsule according to a programmed sequence.

Advantageously, the diffusion of the desired quantity of the perfume or fragrance is selected within a cavity or channel made of or coated on the surface with an adsorbent material, this adsorbent material being selected as to its composition and surface condition to have maximum water-repellency characteristics so that any proportion of the fragrant quantity diffused does not settle on the duct.

Advantageously, the micro-cylinder is fixed on a frame 7] in the example shown with a pyramid shape, attached to a micro-fan 9 responsible for creating an air flow through a duct 13 toward the perfume or fragrant product collector 14.

Advantageously, the device can be refilled from a 110V or 220V mains socket on the one hand and by photovoltaic electricity on the other hand, in particular, by solar cells, which can be positioned on the top of the system or its application models.

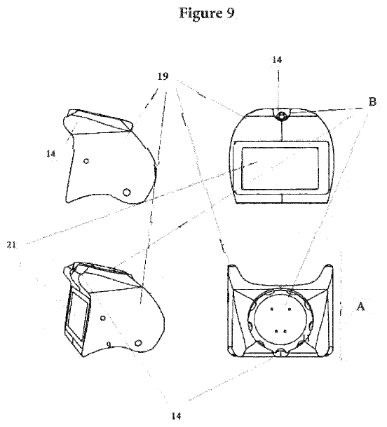

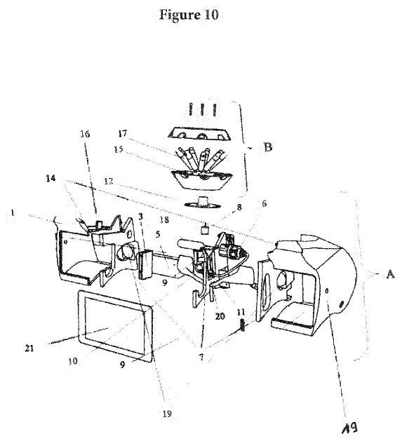

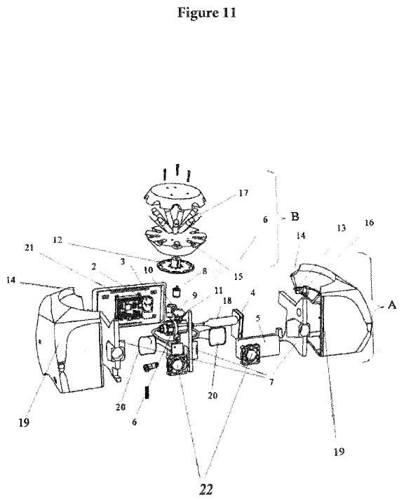

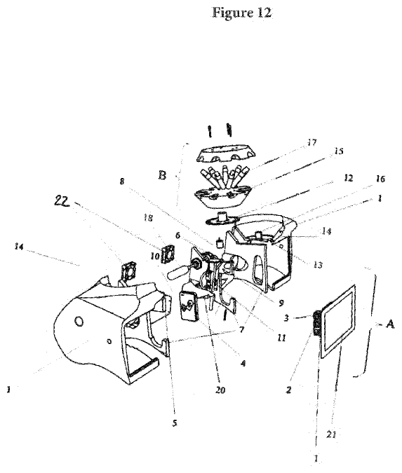

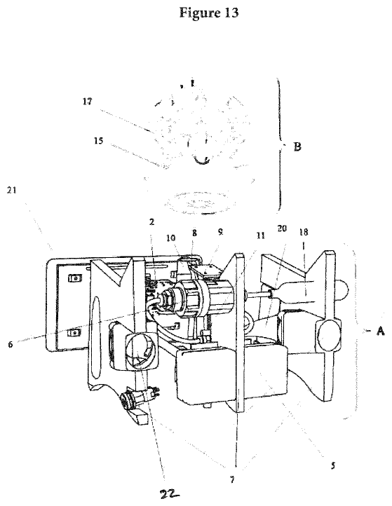

FIGS. 9 to 14 illustrate another embodiment of the diffusion device according to the present disclosure (called “SNIFFY THEATRE—SCENTERTAINMENT”). In this embodiment, the device for diffusing volatile substances into the air, and more particularly functional, is able to select, actuate and then diffuse a perfumed or fragrant liquid encapsulated and placed in at least one refillable or consumable multi-capsule cartridge in an applicative environment for entertainment activities, and, in particular, concert halls, cinemas, opera houses and theatres whose seats will be equipped with the system to allow increased and multi-sensory communication.

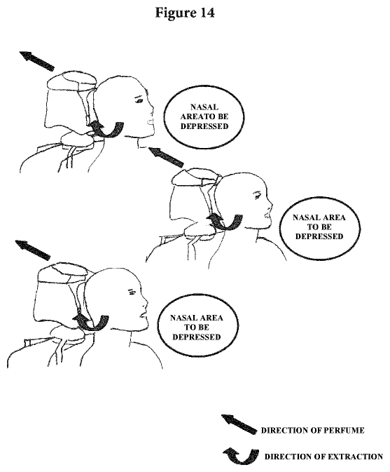

In this embodiment, the diffusion device uses all the characteristics of the device described above. It is different, however, in the arrangement of the components of the part A, divided between three sectional walls of the part A. Furthermore, and according to a preferred version of the present disclosure, the device comprises a pair of air extraction fans 22. This has the advantage of increasing the effectiveness of the device by creating a depression in front of the user's face (FIG. 14).

As before, the air flow created carries the olfactory molecules contained in the chamber/collector that releases the olfactory molecules as close as possible to the nasal apparatus of the customer, collaborator or patient. This targeted micro-nebulization has, in addition to the advantages previously described, namely (i) the management of micro-nebulization, which allows a reduced volume of olfactory liquid to be calibrated with the effect of not saturating the human nasal apparatus, (ii) the micro-nebulization vector placed directly under the nasal apparatus, thus avoiding the effect of olfactory saturation of the atmosphere immediately surrounding the patient/customer/collaborator to help individualize the olfactory experience, and (iii) this non-saturation of the nasal apparatus opens up the possibility of multiple actions over a time slot of variable duration, diminishing or repelling the saturation effect of the human nasal apparatus, the advantage being that the device is equipped with an autonomous apparatus for evacuating polluted/perfumed air by controlling a pair of extraction fans from its processor.

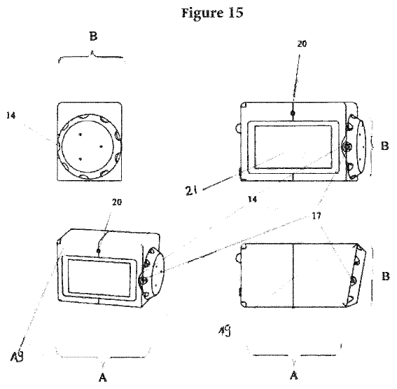

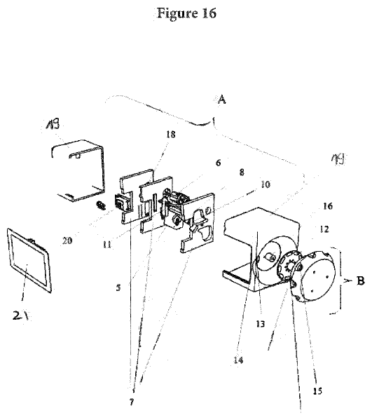

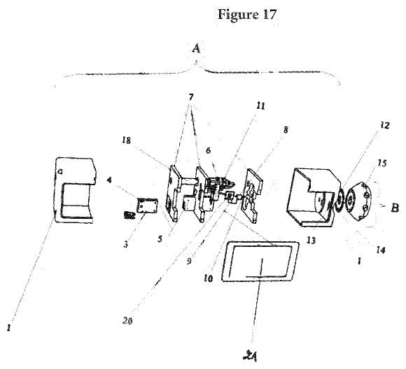

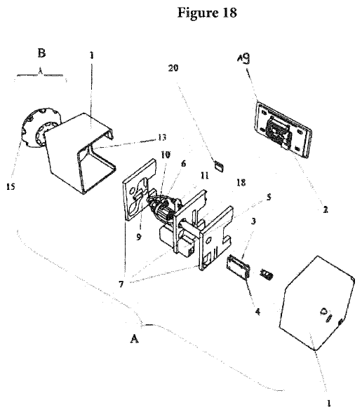

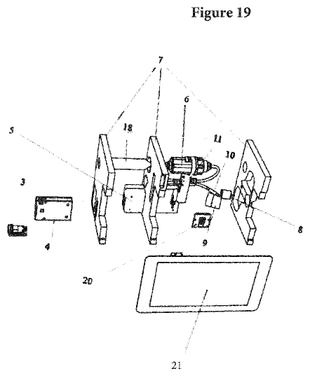

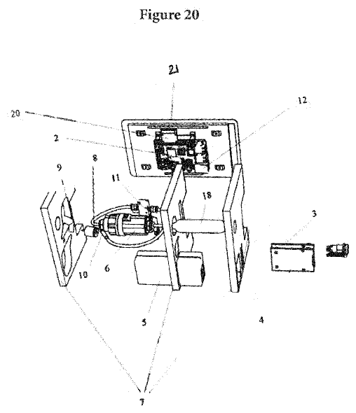

FIGS. 15 to 20 (called “SNIFFY MARKET THEATRE”) illustrate another embodiment of the diffusion device according to the present disclosure. In this embodiment, the device for diffusing volatile substances into the air, and more particularly functional, able to select, activate and then diffuse a perfumed or fragrant liquid encapsulated and designed in at least one refillable or consumable multi-capsule cartridge in an application environment of distribution activities dedicated to the food and beauty industry mainly, and, in particular, through infrastructures such as supermarkets, hypermarkets or specialized supermarkets whose shelves will be equipped with the system to allow increased and multi-sensorial communication as well as instantaneous collection of customer information.

In this embodiment, the diffusion device takes up all the characteristics of the device illustrated in FIGS. 9 to 14. It is different, however, in that it includes a cartridge 18 of compressed air or any other pressure supply to feed the micro-cylinder after passing through a pressure regulator 6 to reduce the output pressure from 30 Bar to a range of 5-7 Bar allowing nominal use of the micro-cylinder, two air extraction fans (not shown) mounted on a perfumed air collector (not shown), a digital touch screen 21 allowing the user to control the usage modes, a camera 20 for collecting behavioral information from the system users.

As before, the air flow created carries the olfactory molecules contained in the chamber/collector that releases the olfactory molecules as close as possible to the nasal apparatus of the customer, collaborator or patient. This targeted micro-emission has, in addition to the advantages previously described, the following advantages: (i) micro-emission management, allowing a reduced volume of olfactory liquid to be calibrated with the effect of not saturating the human nasal apparatus, (ii) the micro-emission vector placed directly under the nasal apparatus, avoiding the effect of olfactory saturation of the atmosphere immediately surrounding the patient/customer/collaborator to help individualize the olfactory experience, and (iii) this non-saturation of the nasal apparatus opens up the possibility of multiple actions over a time slot of variable duration, diminishing or repelling the saturation effect of the human nasal apparatus, the advantage being that the device is equipped with an autonomous apparatus for evacuating polluted/perfumed air by controlling a pair of extraction ventilators from its processor.

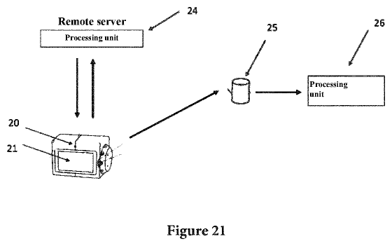

FIG. 21 represents an exemplary architecture of a fragrance diffusion management system using at least one diffusion device according to the present disclosure equipped with at least one associated volatile substance cartridge.

In the illustrated embodiment, the management system comprises, in addition to a diffusion device equipped with a volatile substance cartridge, a first processing unit hosted by a remote server 24, which the diffusion device is connected to, an image acquisition device designed to enable the acquisition of images (a photo, a film) of the user of the diffusion device, a second processing unit 26 connected to the image acquisition device for processing the acquired images. The second processing unit can be hosted on the same server as the first processing unit or on a separate remote server.

The principle of managing the diffusion of fragrances is as follows.

The person wishing to access a specific fragrance selects it via the digital touch screen 21 when it is integrated in the diffusion device, as is the case for the diffusion device for the second and third embodiments, or via a separate display connected to the diffusion device. In response to these instructions, the first control unit determines the capsule corresponding to the person's command. FIG. 21 shows only one diffusion device with a cartridge. Of course, it is obvious that the management system can include a plurality of diffusion mechanisms. In this case, the first processing unit will also define, upon receipt of the control instructions, the diffusion device and the capsule contained in this diffusion device corresponding to the command. Once the capsule has been determined, the first processing unit transmits the instructions to the control module of the diffusion device, which will control the rotation of the motor coupled to the plate that the cartridge is stowed on until the capsule of the cartridge containing the selected perfume is placed in the position required to be activated by the actuator, in this case, in the example described, positioned along the axis of the micro-cylinder. Once the capsule is positioned, the processing module sends the control instructions to control the actuator actuation. Synchronously with the command of activating the actuator, the control module activates the imaging device, i.e. the camera 20, at the time of the diffusion of the perfume, in order to film the person who requested the diffusion of the perfume. These images, previously stored in an external storage unit 26, are transmitted to the second processing unit 26, which will process these images and, from a compilation of images processed in relation to the selected perfume, will establish via specific software a behavioral profile associated with the selected perfume.

The present disclosure is described above by way of example. It should be understood that the person skilled in the art is able to carry out different embodiments of the invention without departing from the scope of the invention.

1.-22. (canceled)

23. A diffusion device for diffusion of fragrant liquid substances into the surrounding air, the diffusion device comprising:
a holder configured to receive a cartridge comprising at least one capsule containing a fragrant liquid substance to be diffused;
a motor coupled to the holder and configured to rotate the holder;
an actuator designed to act upon the at least one capsule so as to cause diffusion of at least a portion of the fragrant liquid substance contained in the at least one capsule; and
a control module configured to control operation of the motor and rotation of the holder, and to control operation of the actuator.

24. The device of claim 23, wherein the actuator comprises a cylinder configured to axially actuate the at least one capsule, the cylinder comprising a spray-pump assembly, compression of the cylinder causing diffusion of the at least a portion of the fragrant liquid substance contained in the at least one capsule.

25. The device of claim 24, wherein the actuator comprises an electronically controlled micro-cylinder for pressing the at least one capsule into the housing as part of a program of the control module.

26. The device of claim 23, wherein the actuator comprises a vibratory actuator configured to produce ultrasonic vibrations causing atomization of the fragrant liquid substance contained in the at least one capsule and to cause the diffusion of at least a portion of the fragrant liquid substance contained in the at least one capsule.

27. The device of claim 23, further comprising at least one fan configured to pulse hot air or gas within the cartridge.

28. The device of claim 27, further comprising at least one of:
a heating resistor; and/or
a device configured to exert an undulatory stress associating at least two asynchronous frequencies, at least one of which is non-linear.

29. The device of claim 23, further comprising a pair of air extraction fans configured to create a zone of air depression so as to create a scented zone.

30. The device of claim 23, further comprising a module for collecting information from a user and/or at least one sensor, the device configured to use the information to determine a composition of the fragrant liquid substance to be diffused and a duration of diffusion of the fragrant liquid substance.

31. The device of claim 23, further comprising a digital touch screen allowing a user of the device to control operation of the device.

32. The device of claim 23, further comprising a camera configured to acquire information.

33. The device of claim 32, wherein the camera is configured to acquire information about the at least one capsule.

34. The device of claim 23, wherein the device is portable.

35. A volatile substance cartridge configured to be detachably mounted on a diffusion device according to claim 23, the cartridge comprising a plurality of housings each adapted to receive respectively a capsule containing a fragrant liquid substance to be diffused, each housing comprising an outlet opening for the fragrant liquid substance diffused by the capsule placed in the housing in response to action of the actuator of the diffusion device when the cartridge is mounted on the diffusion device.

36. The cartridge of claim 35, wherein each housing is provided with a passage allowing the actuator of the diffusion device to axially actuate the respective capsule, wherein each capsule comprises a tube/spray-pump assembly configured to nebulize the fragrant liquid substance contained in the capsule under the effect of the compression by the actuator.

37. The cartridge of claim 35, further comprising a plurality of channels each respectively having an inlet opening and an outlet opening configured to allow passage of an air flow generated by the device up to the outlet opening of the housing, the inlet and outlet openings of each of the channels being independent of one another.

38. The cartridge of claim 35, further comprising a Radio Frequency Identification (FRID) or a Near Field Communication (NFC) chip.

39. The cartridge of claim 35, wherein the device is configured to collect information from a user and/or at least one sensor, the device configured to use the information to determine a composition of the fragrant liquid substance to be diffused and a duration of diffusion of the fragrant liquid substance.

40. The cartridge of claim 35, wherein at least one of the capsules contains a perfume to be diffused, and at least another of the capsules contains a product neutralizing a fragrance perfume or any other substance.

41. The cartridge of claim 35, further comprising a cavity or a channel within which a quantity of a selected fragrant liquid substance is diffused, wherein surfaces defining the cavity or channel are hydrophobic.

42. A system for managing diffusion of fragrant liquid substances in an environment in response to a control action by a user, comprising:
at least one diffusion device according to claim 23, the at least one diffusion device configured to communicate with a first processing unit of a remote server;
at least one cartridge according to claim 35 detachably mounted on the diffusion device; and
a fragrance diffusion control interface connected to the first processing unit.

43. The system of claim 42, further comprising at least one image acquisition device configured to photograph or film the user at the time of diffusion of a fragrant liquid substance in response to a command to diffuse the fragrant liquid substance, the at least one image acquisition device connected to an image processing unit configured to establish a behavioral profile related to the diffused fragrant liquid substance from an acquired photograph or film.

44. The system of claim 42, wherein the control interface is integrated with the diffusion device.
